(19) United States
(12) Patent Application Publication
(10) Pub. No.: US 2006/0215807 A1
(43) Pub. Date: Sep. 28, 2006

(54) Cassette type radiation image detector and radiation image detecting system

(75) Inventor: Hiromu Ohara, Tokyo (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: Konica Minolta Medical & Graphic, Inc., Tokyo (JP)

(21) Appl. No.: 11/157,499

(22) Filed: Jun. 21, 2005

(30) Foreign Application Priority Data
Mar. 25, 2005 (JP) 2005-089186

(51) Int. Cl. A61B 6/00; G01N 23/04

(52) U.S. Cl. 378/11; 378/62

(57) ABSTRACT

A cassette type radiation image detector having: a driving control unit to control a plurality of driving units; and an operation confirming control unit to confirm an operation of the driving control unit, wherein the cassette type radiation image detector obtains an image information.

### BACKGROUND OF THE INVENTION

1. Field of the Invention

The present invention relates to a cassette type radiation image detector and a radiation image detecting system. In particular, the present invention relates to a cassette type radiation image detector and a radiation image detecting system for radiographing a radiation image represented by an X-ray image.

2. Description of Related Art

So far, in a medical diagnosis, a radiation image which is obtained by irradiating a radiation such as an X-ray or the like to a subject and by detecting an intensity distribution of the radiation transmitted through the subject has been widely in use. These days, a radiation image generating apparatus using an FPD (Flat Panel Detector; radiation image detector) that upon an image radiation, detects a radiation and converts the detected radiation into an electric energy to be detected as a radiation image information is being proposed. In such a radiation image generating apparatus, a diagnosis function for checking a state of an image generating unit is provided for judging whether the image generation has been performed normally or not (for example, see JP-Tokukai-2004-73489A).

Further, these days, a cassette-type FPD in which the FPD is contained in a cassette for the purpose of improving a transportability and a handling ability of the FPD is being developed (for example, see JP-Tokukaihei-6-342099A). In particular, in order to utilize the transportability of the FPD, a cassette-type FPD which wirelessly communicates with a console for a control thereof is being proposed.

The above-mentioned diagnosis function is to check an operation of only the image generation unit. In this diagnosis mode, under a specific X-ray exposure condition, in order to judge whether an image generation is performed with a desired certain dose over the whole part of a light receiving unit of the X-ray image generating apparatus or not, checking of a pixel output value is performed for the evaluation to see whether a pixel output value within a. requirement has been obtained. However, by the conventional diagnosis function, it is not possible to check an operation of the cassette-type FPD. In other words, even when the image generation unit normally functions, it is not possible to obtain a correct image if the cassette-type FPD is not normal, and therefore an image generation needs to be re-performed. Fewer times of re-performing of an image generation are more desirable in order not to give unnecessary exposure to a patient.

In particular, since the cassette-type FPD is transportable, it is used in various locations such as an image generation room, a ward, an operating room and the like, without staying in a specific place to be used for an image generation. In other words, it is necessary to check the communication state at each location.

### SUMMARY OF THE INVENTION

An object of the present invention is to make it possible to perform an operation check at various locations and to prevent an unnecessary re-performance of an image generation by making it possible to perform an operation confirmation by a cassette type radiation image detector itself.

In order to accomplish the above-mentioned object, in accordance with a first aspect of the present invention, the cassette type radiation image detector comprises:

a driving control unit-to control a plurality of driving units; and

an operation confirming control unit to confirm an operation of the driving control unit,

wherein the cassette type radiation image detector obtains an image information.

According to the first aspect, the detector comprises an operation confirming control unit for confirming an operation of a driving control unit. Therefore, it is possible to confirm an operation by the cassette type radiation image detector itself. Thereby, it is possible to confirm the operation at various locations. Then, if it is possible to confirm the operation by the cassette type radiation image detector itself, it is possible to enhance the accuracy of the image generation, and as a result it is possible to prevent unnecessary re-performance of the image generation.

Moreover, the detector may further comprises a battery as an electric power supplying source to the plurality of driving units, the driving control unit and the operation confirming control unit,

wherein the driving control unit comprises a battery residual quantity detecting unit to detect a residual quantity of the battery.

Moreover, the plurality of driving units may comprise a storing unit to store the image information, and

the driving control unit may comprise a storing operation detecting unit to detect whether or not a storing operation of the storing unit is appropriate.

Moreover, the plurality of driving units may comprise a communication unit to communicate with an external device, and

the driving control unit may comprise a communication operation detecting unit detecting whether or not a communication operation of the communication unit is appropriate.

Moreover, the operation confirming control unit may comprise a driving control appropriateness judging unit to judge whether or not the driving control unit is normally operated.

Moreover, the driving control appropriateness judging unit may judge whether the driving control unit is operated normally based on detected results of the battery residual quantity detecting unit, the storing operation detecting unit and the communication operation detecting unit of the driving control unit.

Moreover, the detector may further comprise a reporting unit to report based on a control of the operation confirming control unit,

wherein the operation confirming control unit may control the reporting unit to report an abnormality when the driving control appropriateness judging unit judges that the driving control unit is not operated normally.

Moreover, the driving control unit may comprise an operation confirming control appropriateness judging unit to judge whether or not the operation confirming control unit is operated normally.

Moreover, the detector may further comprise a reporting unit to report based on a control of the driving control unit,

wherein the driving control unit controls the reporting unit to report an abnormality when the operation confirming control appropriateness judging unit judges that the operation confirming control unit is not operated normally.

In accordance with a second aspect of the present invention, a radiation image detecting system comprises:

a cassette type radiation image detector to obtain an image information, having a driving control unit to control a plurality of driving units, and an operation confirming control unit to confirm an operation of the driving control unit; and

a console to control the cassette type radiation image detector.

Moreover, the operation confirming control unit may control the driving control appropriateness judging unit to transmit an information indicating an abnormality to the console, when the driving control appropriateness judging unit judges that the driving control unit is not operated normally.

Moreover, the driving control unit controls the operation confirming control appropriateness judging unit to transmit an information indicating an abnormality to the console, when the operation confirming control appropriateness judging unit judges that the operation confirming control unit is not operated normally.

### BRIEF DESCRIPTION OF THE DRAWINGS

The present invention will become more fully understood from the detailed description given hereinafter and the accompanying drawing given by way of illustration only, and thus are not intended as a definition of the limits of the present invention, and wherein:

FIG. 1 is a view showing a schematic structure representing one embodiment of a radiation image detecting system relating to the present invention,

FIG. 2 is a perspective view showing a substantial structure of a cassette type radiation image detector relating to the present invention,

FIG. 3 is a block diagram showing a substantial structure of the cassette type radiation image detector relating to the present invention,

FIG. 4 is a view showing an equivalent circuit of a photoelectrical conversion unit for processing one pixel, structuring a signal detecting unit provided in the cassette type radiation image detector of FIG. 2,

FIG. 5 is a view showing an equivalent circuit in which the photoelectrical conversion units of FIG. 4 are arranged two-dimensionally, and

FIG. 6 is a block diagram showing a substantial structure of a console structuring the radiation image detecting system of FIG. 1.

### PREFERRED EMBODIMENT OF THE INVENTION

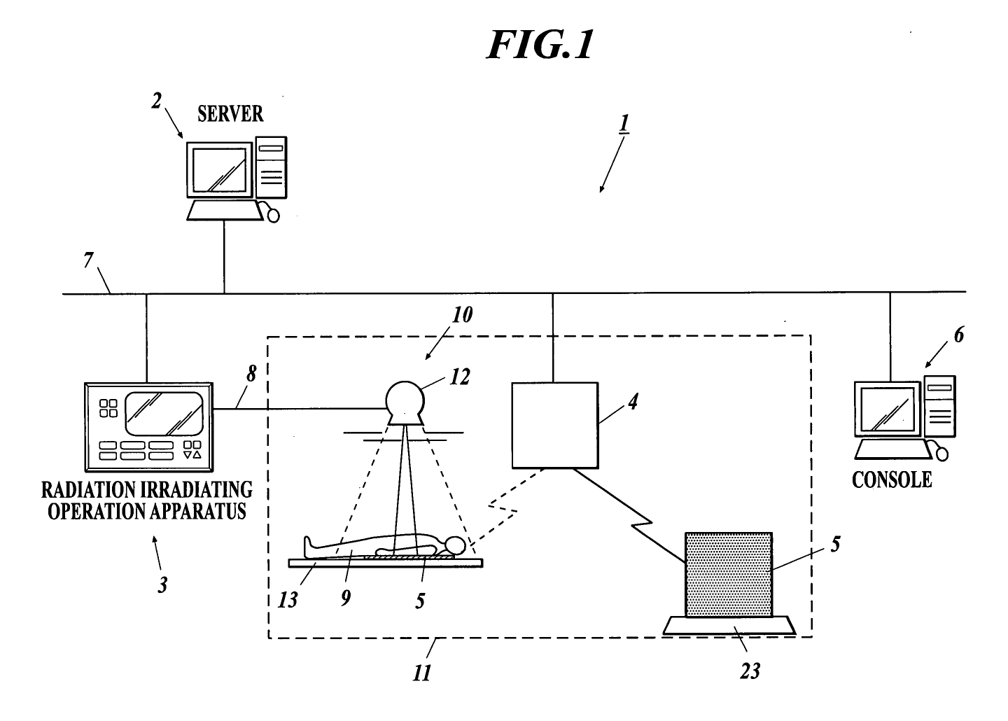

Hereinafter, an embodiment of the present invention will be described with reference to FIG. 1 to FIG. 6. FIG. 1 is a view showing a schematic structure representing one embodiment of a radiation image detecting system relating to the present invention.

A radiation image detecting system 1 in the present embodiment is, for example, a system which is applied to a radiation image generation performed in a hospital. As shown in FIG. 1, in the radiation image detecting system 1, a server 2 for managing various information regarding radiography, a patient an the like, a radiation irradiating operation apparatus 3 for performing an operation regarding a radiation image generation, a base station 4 for establishing a communication according to a wireless communication such as a wireless LAN (Local Area Network) or the like, and a console 6 for controlling a cassette type radiation image detector 5 and for performing an image processing or the like to a radiation image detected by the cassette type radiation image detector 5 are connected through a network 7. A radiation image generating apparatus 10 for generating a radiation image by irradiating a radiation to a patient being a subject 9 is connected to the radiation irradiating operation apparatus 3 through a cable 8. For example, one pair of the radiation image generating apparatus 10 and the cassette type radiation image detector 5 is installed in each image generation room 11. The radiation image generating apparatus 10 is operated by the radiation irradiating operation apparatus 3 and the radiation image is detected by the cassette type radiation image detector 5, whereby it is possible to obtain radiation image information. Here, a plurality of cassette type radiation image detectors 5 may be provided in one image generation room 11.

Here, the network 7 may be a communication line dedicated to the system. However, it is preferable to use an existing line such as Ethernet (trademark) or the like, because the dedicated communication line may decrease a flexibility of the system structure, or the like. Incidentally, in addition to the components mentioned above, a plurality of radiation irradiating operation apparatuses 3 for operating the radiation image generating apparatuses 10 in other image generation rooms 11, a plurality of cassette type radiation image detector 5 and a plurality of consoles 6 may be connected to the network 7.

First, the radiation irradiating operation apparatus 3 comprises an input operation unit (not shown) comprising an operation panel and the like for operating the radiation image generating apparatus 10 for inputting an image generation condition, for example, a tube voltage, a dose (mAs value) and the like, a display unit (not shown) for displaying an information such as the image generation condition and the like, and various instructions, etc, a power source (not shown) for supplying an electrical power to the radiation image generating apparatus 10, and the like.

The radiation image generating apparatus 10 is installed at the inside of the image generation room 11, and comprises a radiation source 12 for irradiating a radiation such as an X-ray or the like. The radiation is irradiated from the radiation source 12 at a tube voltage and a dose set by the radiation irradiating operation apparatus 3. As the radiation source 12, for example, a radiation tube is used. The radiation tube accelerates an electron generated according to a thermal excitation by a high voltage, to be crashed to a cathode, whereby a radiation is generated.

Next, the cassette type radiation image detector 5 detects a radiation irradiated from the radiation source 12 of the radiation image generating apparatus 10 and transmitted through the subject 9, for obtaining a radiation image. The cassette type radiation image detector 5 is located within an irradiation area of the radiation that is irradiated from the radiation source upon the image generation. Here, as shown in FIG. 1, the cassette type radiation image detector 5 is, for example, located between the subject 9 and a platform 13 on which the subject is to be put. However, the location of the cassette type radiation image detector 5 is not limited to such a location. For example, it may be such that a detector attaching portion (not shown) to which the cassette type radiation image detector 5 is to be attached is provided below the platform, and the cassette type radiation image detector 5 is attached to the detector attaching portion.

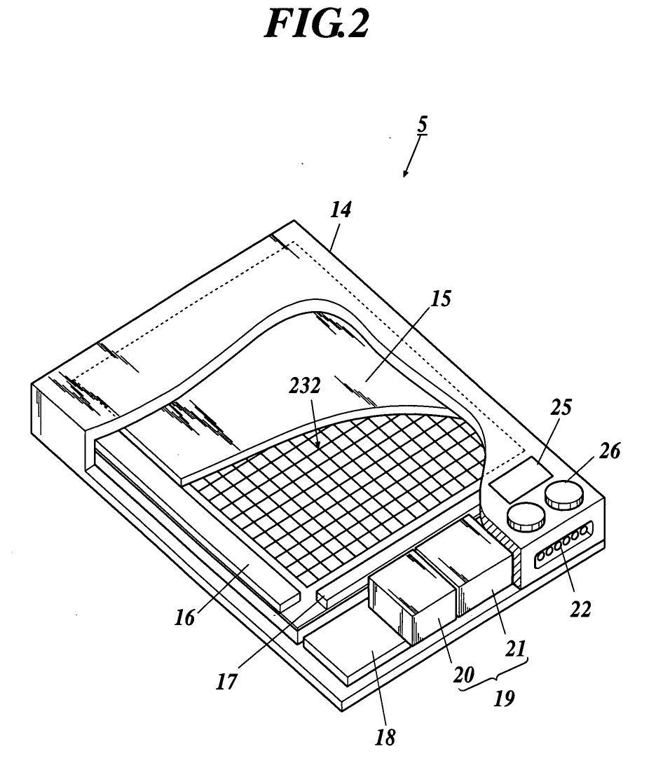

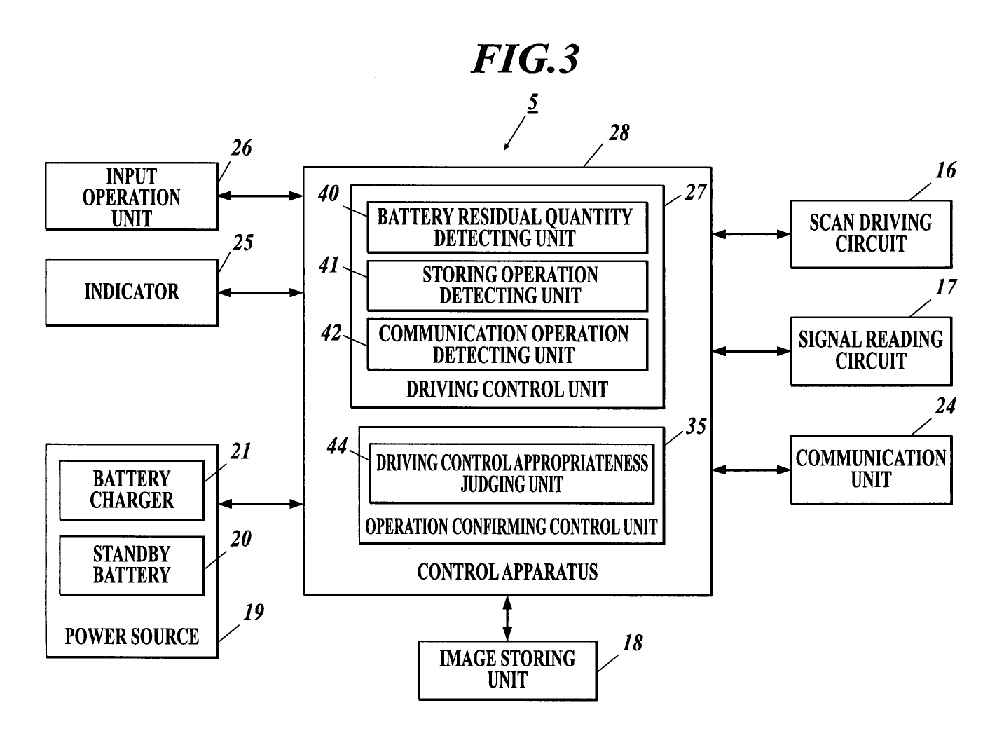

The cassette type radiation image detector 5 is a cassette type radiation image detector 5 being a cassette-type flat panel detector. Hereinafter, with reference to FIG. 2 and FIG. 3, a structure of the cassette type radiation image detector 5 will be described.

As shown in FIG. 2, the cassette type radiation image detector 5 comprises a chassis 14 for protecting the inside thereof. The cassette type radiation image detector 5 is structured to be portable as a cassette.

At the inside of the chassis 14, an imaging panel 15 for converting the irradiated radiation into an electric signal is so formed as to structure a layer. At a side of the imaging panel 15 where the radiation is irradiated, a luminescent layer (not shown) for emitting a light according to an intensity of the entered irradiation is provided.

The luminescent layer is in general called a scintillator layer. For example, the luminescent layer has a phosphor as a main component, and based on the entered radiation, the luminescent layer outputs an electromagnetic wave (light) having a wavelength from 300 nm to 800 nm, that is, an electromagnetic wave from an ultraviolet light through a visible light to an infrared light.

As a phosphor used in the luminescent layer, for example, a material having a parent body of CaWO₄ or the like, or a material having a parent body of Csl;Tl, Gd₂O2ₛ:Tb, TnS:Ag or the like in which a luminescence center material is activated can be used. Further, with a rare earth element defined as M, a phosphor represented by a general formula (Gd, M, Eu)₂O₂ can be used. In particular, it is preferable to use Csl:Tl or Gd₂O₂S:Tb since Csl:Tl and Gd₂O₂S:Tb have a high radiation absorbability and a high luminescence efficiency. By using Csl:Tl or Gd₂O₂S:Tb, it is possible to obtain a high quality image without too much noise.

On a surface being an opposite side to the surface to which the radiation of the luminescent layer is irradiated, formed is a signal detecting unit 232 for converting the electromagnetic wave (light) outputted from the luminescent layer into an electric energy to be accumulated and for outputting an image signal based on the accumulated electric energy.

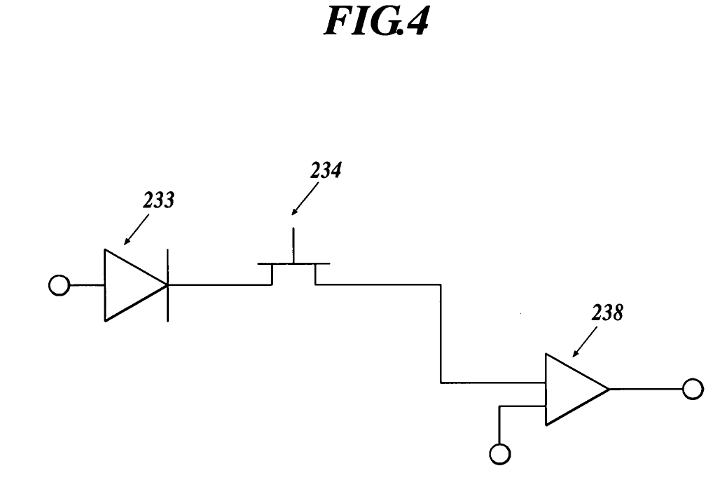

Here, a circuit structure of the imaging panel 15 will be described. FIG. 4 is a view showing an equivalent circuit of the photoelectric conversion unit for processing one pixel, structuring the signal detecting unit 232.

As shown in FIG. 4, the photoelectric conversion unit for processing one pixel comprises a photodiode 233, and a thin film transistor (hereafter, TFT 234) for picking up the electric energy accumulated in the photodiode 233 according to a switching. The picked-up electric signal is amplified by an amplifier 238 to a level at which a signal reading circuit 237 is capable of detecting. Here, the TFT 234 and a reset circuit (not shown) structured from a condenser are connected to the amplifier 238, and a reset operation for resetting the accumulated electric signal is performed at the reset circuit by switching the TFT 234 on. Further, the photodiode 233 may simply be a light condenser comprising a parasitic capacitance, or may be one including a juxtaposition of additional condensers so as to improve a dynamic range of the photodiode 233 and the photoelectric conversion unit. Here, the photodiode 233 may be one using an inorganic semiconductor system or one using an organic semiconductor system.

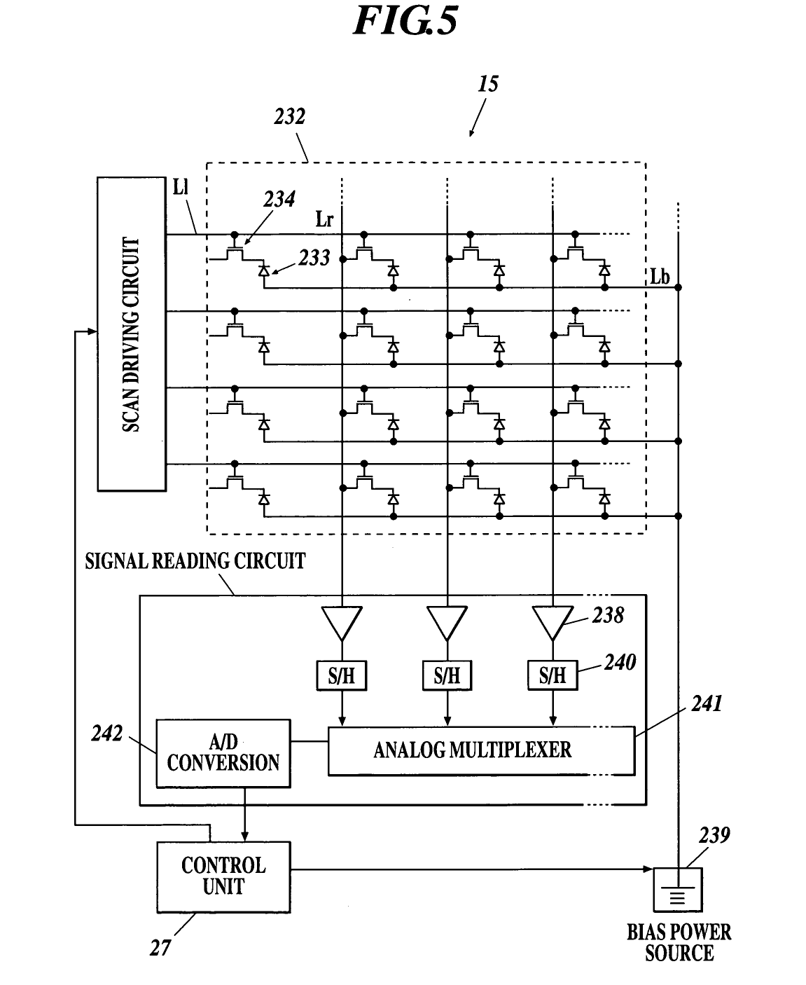

FIG. 5 is a view showing an equivalent circuit in which such photoelectrical conversion units are arranged two-dimensionally, and a scanning line Ll and a signal line Lr are so arranged to be perpendicular to each other between the pixels. The TFT 234 is connected to the mentioned photodiode 233, and an edge of the photodiode 233 to which the TFT 234 is connected is connected to the signal line Lr. Meanwhile, another edge of the photodiode 233 is connected to edges of photodiodes 233 being adjacent to each other, and is connected to a bias power source 239 through a common bias line Lb. An edge of the bias power source 239 is connected to a driving control unit 27, and a voltage is applied to the photodiode 233 through the bias line Lb according to an instruction from the driving control unit 27. Further, the TFTs 234 arranged on each row are connected a common scan line Ll, and the scan line Ll is connected to the driving control unit 27 through a scanning driving circuit 236. Similarly, the photodiodes 233 arranged on each column are connected to the common signal line Lr to be connected to the signal reading circuit 237 controlled by the driving control unit 27. In the signal reading circuit 237, provided on the common signal line Lr are the amplifier 238, a sample-and-hold circuit 240, an analog multiplexer 241 and an A/D converter 242, in the order from the one closest to the imaging panel 15.

Here, the TFT 234 may be one using an inorganic semiconductor system such as the one used in a liquid crystal display or the like, or one using an organic semiconductor system.

Further, in the present embodiment, a case in which the photodiode 233 as the photoelectrical conversion device is used is described. However, a CCD other than the photodiode may be used as the photoelectrical conversion device.

At the side of the signal detecting unit 232, provided are a scan driving circuit 16 for scanning and driving each photoelectrical conversion device by transmitting a pulse to each photoelectrical conversion device, and a signal reading circuit 17 for reading out an electric energy accumulated in each photoelectrical conversion device.

Further, as shown in FIG. 2 and FIG. 3, the cassette type radiation image detector 5 comprises an image storing unit 18 comprising a rewritable memory such as a nonvolatile memory (RAM), a flash memory or the like. The image storing unit 18 stores the image signal outputted from the imaging panel 15. The image storing unit 18 may be an incorporated-type memory, or a detachable memory such as a memory card.

Further, in the cassette type radiation image detector 5, a power source 19 is provided as an electric power supplying source for supplying an electric power to a plurality of driving units (the scan driving circuit 16, the signal reading circuit 17, a communication unit 24 (described later), the image storing unit (storing unit) 18, a control apparatus 28, a battery residual quantity detecting unit 40 (described later), an indicator 25 (described later), an input operation unit 26 (described later), the imaging panel 15 and the like) structuring the cassette type radiation image detector 5. The power source 19 comprises a standby battery 20 such as a manganese battery, an alkaline battery, an alkaline button battery, a lithium battery, a silver oxide battery, a zinc air battery, a nickel-cadmium battery, a mercury battery, a lead-acid battery or the like, and a chargeable battery charger (battery) 21 such as a nickel cadmium battery, a nickel hydrogen battery, a lithium ion battery, a small-type sealed lead acid battery, a lead accumulator, a fuel battery, a solar battery or the like. In this way, by comprising the standby battery 20 in addition to the battery charger 21, it is possible to supply at least a minimum electric power to the cassette type radiation image detector 5 even when the battery charger 21 is short of charged amount or even while the battery charger 21 is being changed. Therefore, it is possible to avoid erasing image information stored in the image storing unit 18 by mistake, or to avoid a state in which it is not possible to receive a signal from an external apparatus such as the console 6 or the like.

At an edge of the chassis 14, a terminal 22 for a charging purpose is formed. For example, as shown in FIG. 1, by attaching the cassette type radiation image detector 5 to a charging device 23 such as a cradle or the like, a terminal of the charging device 23 side and the terminal 22 of the chassis side are connected, and charging of the battery charger 21 is performed. Further, the battery charger 21 is exchangeable by taking it from a side part of the chassis 14. In addition, forms of the standby battery 20 and the battery charger 21 structuring the power source 19 are not limited to what is shown in FIG. 2. For example, a battery in a plate shape may be provided in parallel with the imaging panel 15. When each battery is provided in such a form, a ratio of the imaging panel surface with respect to the chassis 14 increases, and thereby it is possible to increase an effective imaging area. Therefore, it is possible to reduce a whole size of the cassette type radiation image detector 5 while the imaging area remains the same. As a result, it is possible to miniaturize the cassette type radiation image detector 5.

Further, in the cassette type radiation image detector 5, the communication unit 24 (see FIG. 3) for transmitting/receiving various signals to/from an external apparatus such as the console 6 and the like, is provided. The communication unit 24, for example, transmits the image signal outputted from the imaging panel 15 to the console 6 and receives an image generation instruction signal, a standby instruction signal and the like transmitted from the console 6.

Further, at one edge of the surface of the chassis 14, the indicator (reporting unit) 25 for displaying a charging status of the battery charger 21, various operation statuses and the like to be reported is provided. Thereby, it is possible for an operator to visually confirm the charging status of the battery charger 21 or the like of the cassette type radiation image detector 5.

At the outside of the chassis 14, the input operation unit 26 for inputting an image generation instruction and a standby instruction is provided.

Further, as shown in FIG. 3, the cassette type radiation image detector 5 comprises the control apparatus 28 comprising the driving control unit 27 and an operation confirming control unit 35 each comprising a CPU, a ROM, a RAM and the like (none of them is shown).

The driving control unit 27 reads out a predetermined program stored in the RAM and develops the read program into an operation area of the RAM. Then, the CPU executes various processes according to the program. Thereby, the driving control unit 27 controls the plurality of driving units provided in the cassette type radiation image detector 5. The driving control unit 27 comprises a battery residual quantity detecting unit 40 for detecting a residual quantity of the battery 21, a storing operation detecting unit 41 for detecting whether or not a storing operation of the image storing unit 18 is appropriate, and a communication operation detecting unit 42 for detecting whether or not a communication operation of the driving control unit 27 is appropriate. Here, the driving control unit 27 may confirm an operation of each driving unit other than these driving units.

The battery residual quantity detecting unit 40 detects a residual quantity of the battery charger 21 based on a control of the driving control unit 27, and outputs the obtained battery residual quantity to the operation confirming control unit 35. It is possible to consider various timings for detecting the battery residual quantity. In the present embodiment at least, when an instruction to switch from a standby state to an image generation capable state (image generation instruction) is inputted from the input operation unit 26 or the communication unit 24, the driving control unit 27 controls the battery residual quantity detecting unit 40 to detect a residual quantity of the battery charger 21. Here, the standby state is a state in which an electric power consumption is less than that of the image generation capable state.

In the ROM of the driving control unit 27, various control data is stored in addition to the programs. The control data comprises, for example, residual quantity judging data for judging whether a residual quantity of the battery charger 21 satisfies a quantity with which an image generation is possible.

The storing operation detecting unit 41 detects whether or not a storing operation of the image storing unit 18 is appropriate based on the control of the driving control unit 27, and outputs the detection result to the operation confirming control unit 35. The detection of the appropriateness of the storing operation is performed so that, for example, a check signal for requesting a reply is transmitted from the storing operation detecting unit 41 to the image storing unit 18, and when there is a reply from the image storing unit 18, the storing operation detecting unit 41 judges that the storing operation is normal (appropriate), or when there is not a reply, the storing operation detecting unit 41 judges that the storing operation is abnormal (inappropriate).

The communication operation detecting unit 42 detects whether a communication operation of the communication unit 24 based on the control of the driving control unit 27, and outputs the detection result to the operation confirming control unit 35. The detection of the appropriateness of the communication operation is performed so that, for example, a check signal for requesting a reply is transmitted to the communication operation detecting unit 41 to the communication unit 24, and when there is a reply from the communication unit-24, the communication operation detecting unit 42 judges that the communication operation is normal (appropriate), or when there is not a reply, the communication operation detecting unit 42 judges that the communication operation is abnormal (inappropriate).

Then, based on the result of the residual quantity detection upon the input of an image generation instruction from the input operation unit 26 or the communication unit 24, the driving control unit 27 switches a state between the image generation capable state and the standby state.

Then, information inputted from the input operation unit 26 and/or a signal received from the communication unit 24 is transmitted to the driving control unit 27, and the driving control unit 27 controls each part based on the transmitted information and signal.

Further, the driving control unit 27 drives the scan driving circuit 16 to transmit a pulse to each photoelectric conversion device, for making each photoelectric conversion device scan and for driving each photoelectric conversion device. Then, the signal reading circuit 17 for reading out an electric energy accumulated in each photoelectric conversion device reads out an image signal, and the read image signal is transmitted to the driving control unit 27. The driving control unit 27 stores the transmitted image signal in the image storing unit 18. Further, the image signal stored in the image storing unit 18 is transmitted to the console 6 through the communication unit 24 accordingly.

The operation confirming control unit 35 reads out a predetermined program stored in the ROM and develops the read program into an operation area of the RAM. Then, the CPU executes various processes according to the program for confirming an operation of the driving control unit 27. The operation confirming control unit 35 comprises a driving control appropriateness judging unit 44 for judging whether or not an operation of the driving control unit 27 is normal (appropriate) based on the detection results of the battery residual quantity detecting unit 40, the storing operation detecting unit 41 and the communication operation detecting unit 42 inputted from the driving control unit 27. Here, the judgment of whether or not the operation of the driving control unit 27 is normal is performed so that when at least one of the detection results of the battery residual quantity detecting unit 40, the storing operation detecting unit 41 and the communication operation detecting unit 42 is abnormal, the driving control appropriateness judging unit 44 judges that the operation of the driving control unit 27 is abnormal.

Next, an operation confirming by the operation confirming control unit 35 will be described.

The operation confirming control unit 35 confirms an operation of the driving control unit 27 at a predetermined timing, such as when an instruction to switch a state to the image generation capable state is made, when a certain instruction is inputted from the console 6, when a certain period has passed since the previous operation confirming is performed, or the like.

Upon the timing of the operation confirming, the operation confirming control unit 35 transmits a signal for the operation confirming to the driving control unit 27, and by judging whether or not the signal is replied from the driving control unit 27, when there is a reply, the operation confirming control unit 35 judges that the operation of the driving control unit 27 is normal, or when there is not a reply, the operation confirming control unit 35 judges that the operation of the driving control unit 27 is abnormal. Here, when the driving control unit 27 receives the signal for the operation confirming, the battery residual quantity detecting unit 40, the storing operation detecting unit 41 and the communication operation detecting unit 42 perform the respective detections, and the results of the detections are transmitted to the driving control appropriateness judging unit 44 of the operation confirming control unit 35. The driving control appropriateness judging unit 44 judges whether or not the operation of the driving control unit 27 is normal based on each of the detection results. When it is judged that an abnormality occurs, the operation confirming control unit 35 controls the indicator 25 to display information indicating abnormality occurrence and a spot of the abnormality occurrence to the console 6. Further, the driving control unit 27 controls to transmit a signal indicating that the abnormality occurred and a spot of the abnormality occurrence to the console 6 through the communication unit 24.

Further, when the residual quantity detection result is inputted to the operation confirming control unit 35 from the battery residual quantity detecting unit 40, the operation confirming control unit 35 confirms an operation of the battery charger 21 based on the inputted result, and the content of the confirmation is displayed on the indicator 25. At this time, when the-residual quantity detection result is less than a quantity with which an image generation is possible, the operation confirming control unit 35 controls the indicator 25 to display information indicating that an image generation is impossible. Further, the operation confirming control unit 35 transmits the corresponding signal to the console 6 through the communication unit 24.

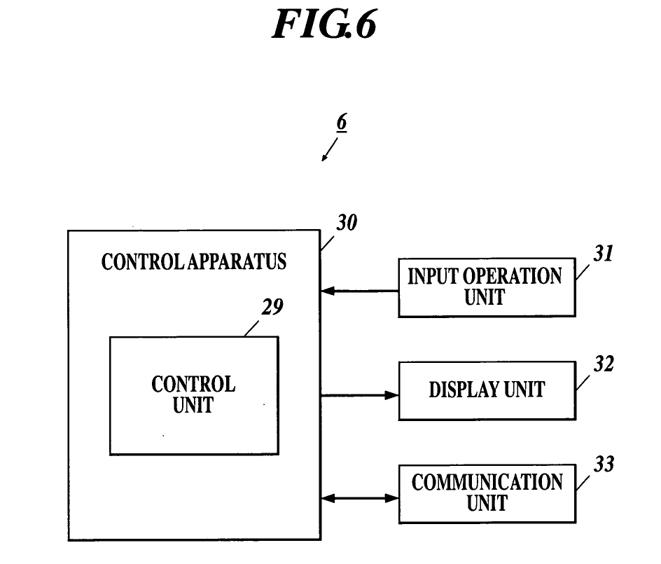

Next, as shown in FIG. 6, the console 6 comprises, for example, a control apparatus 30 comprising a control unit 29 comprising a generic CPU, a generic ROM, a generic RAM and the like (none of them is shown). The control unit 29 reads out a predetermined program stored in the ROM and develops the read program into an operation area of the RAM, and the CPU executes various processes according to the program.

Further, the console 6 comprises an input operation unit 31 for inputting various instructions and the like, a display unit 32 for displaying an image, various messages and the like, a communication unit 33 for transmitting/receiving a signal to/from an external device such as the cassette type radiation image detector 5 and the like, etc.

The input operation unit 31 comprises, for example, an operation panel, a keyboard, a mouse and the like. The input operation unit 31 outputs a pushed signal of a key pushed on the operation panel or the keyboard by an operation to the control unit 29 as an input signal.

The display unit 32 comprises a CRT (Cathode Ray Tube), an LCD (Liquid Crystal Display) or the like, and displays various screens according to an instruction of a display signal outputted from the control unit 29.

The communication unit 33 is to communicate various information with the cassette type radiation image detector 5 through the base station 4 according to a wireless communication system such as a wireless LAN or the like.

To the control unit 29, a signal inputted from the input operation unit 31, a signal received from outside through the communication unit 33 and the like are transmitted, and the control unit 29 performs a predetermined process corresponding to the transmitted signal. For example, radiation image information detected by the cassette type radiation image detector 5 is transmitted to the control unit 29, and the control unit 29 performs a predetermined image process based on the transmitted radiation image information for obtaining a radiation image. Further, the control unit 29 displays on the display unit 32, a radiation image, a thumbnail image, various information inputted from the input operation unit 31, a signal indicating that an abnormality of the cassette type radiation image detector 5 occurs, a residual quantity of the battery charger 21 based on a detection result by the battery residual quantity detecting unit 40, a state of the cassette type radiation image detector 5 (image generation capable state or standby state) and the like.

Next, an operation of the radiation image detecting system 1 to which the cassette type radiation image detector 5 relating to the present embodiment is applied will be described.

Normally, in a state where an image generation reservation is not inputted to the cassette type radiation image detector 5, in order to perform an image generation as soon as the reservation is made, the driving control unit 27 of the cassette type radiation image detector 5 controls each operation state of the plurality of driving units to be in the standby state.

Thereafter, when the image generation reservation instruction is inputted to the console 6, the radiography technician (operator) selects one of the cassette type radiation image detector 5 to be used for the image generation on the console 6, and inputs accordingly to the input operation unit 31. The inputted content is transmitted to the communication unit 24 of the selected cassette type radiation image detector 5 through the communication unit 33 of the console 6, and inputted to the control unit 27, for example, as image generation instruction information. The driving control unit 27 controls an electrical power consumption quantity of the battery charger 21 to switch a state from the standby state to the image generation state. Before the state is switched, the driving control unit 27 controls the battery residual quantity detecting unit 40 to detect a residual quantity of the battery charger 21. Here, even when the radiography technician directly operates the input operation unit 26 of the cassette type radiation image detector 5 to input an image generation instruction, the driving control unit 27 controls an operation state of each of the plurality of driving units based on the image generation instruction, to switch a state from the standby state to the image generation state by controlling an electric power consumption quantity of the battery charger 21.

When the switching signal is inputted to the operation confirming control unit 35, the operation confirming control unit 35 recognizes the switching signal as an operation confirming timing, the operation confirming control unit 35 controls the driving control appropriateness judging unit 44 to confirm an operation of the driving control unit 27. When an abnormality is not detected according to a result of confirming the operation, the operation confirming control unit 35 transmits information indicating that there is no abnormality to the driving control unit 27, and the image generation control thereafter is continuously performed. On the other hand, when an abnormality is detected, the operation confirming control unit 35 controls the indicator 25 to display information indicating that the abnormality occurs. At the same time, when the communication unit 24 is normally operated, the operation confirming control unit 35 transmits the information that the abnormality occurs to the console 6 through the communication unit 24. The control unit 29 of the console 6 controls the display unit 32 to display information indicating that an abnormality of the cassette type radiation image detector 5 occurs.

In response to the content displayed on the indicator 25 of the display unit 32 of the console 6 or the indicator 25 of the cassette type radiation image detector 5, the radiography technician determines a coping method corresponding to the abnormality.

When an abnormality occurs to the driving control unit 27, for example, an instruction to prompt a reset of the driving control unit 27 is inputted to the input operation unit 31. Thereby, the reset signal is transmitted to the operation confirming control unit 35 through the communication unit 33 of the console 6, and the operation confirming control unit 35 resets the driving control unit 27. Here, a judgment of the reset of the driving control unit 27 may be done by the operation confirming control unit 35 by itself.

When an abnormality occurs to the image storing unit 18 or the communication-unit 24, for example, repairs, replacement and the like can be expected as a coping method. When an abnormality occurs to battery charger 21, for example, repairs, charging and the like can be expected as a coping method.

As above, in accordance with the present embodiment, since the operation confirming control unit 35 for confirming an operation of the driving control unit 27 is provided, it is possible to confirm an operation of the driving control unit 27 by the cassette type radiation image detector 5 by itself. Thereby, it is possible to confirm the operation at various locations. Then, if it is possible to confirm the operation by the cassette type radiation image detector 5 by itself, it is possible to enhance an accuracy of an image generation. Consequently, it is possible to prevent an unnecessary re-performance of an image generation.

Further, since the operation confirming control unit 35 confirms the operation of the battery charger 21, it is possible to avoid a situation in which it is not possible to perform an image generation due to lack of a battery residual quantity.

Further, since the operation confirming control unit 35 confirms the operation of the image storing unit 18, it is possible to prevent an image generation when the operation of the image storing unit 18 is abnormal.

Then, since the operation confirming control unit 35 confirms the operation at a predetermined timing, it is possible to automatically confirm the operation even without giving an instruction to confirm the operation.

Further, when an abnormality is detected by confirming the operation by the operation confirming control unit 35, the abnormality is reported by the indicator 25. Therefore it is possible to report the occurrence of the abnormality to a radiography technician. Thereby, it is possible to cope with the abnormality immediately.

Further, when an abnormality of the communication unit 24 is detected, the abnormality is reported by the indicator 25. Therefore, it is possible to report the occurrence of the abnormality to the radiography technician. Thereby, it is possible to cope with the abnormality of the communication unit 24 immediately.

Then, when an abnormality is detected by the operation confirming control unit 35 confirming the operation, if the operation of the communication unit 24 of the cassette type radiation image detector 5 is normal, information indicating the abnormality is transmitted to the console 6 through the communication unit 24. Thereby, it is possible to confirm whether there is an abnormality at a side of the console 6.

Here, the present invention is not limited to the above-mentioned embodiment, and can be changed accordingly.

For example, in the present embodiment, the operation confirming control unit 35 confirms the operation of the driving control unit 27. However, the driving control unit 27 may further confirm the operation of the operation confirming control unit 35. Thereby, since it is possible to detect an abnormality of the operation confirming control unit 35 itself, it is possible to enhance an accuracy of confirming the operation. Concretely, an operation confirming control appropriateness judging unit for judging whether or not the operation confirming control unit 35 is operated normally is provided at the driving unit 27. The operation confirming control appropriateness judging unit judges whether or not the operation confirming control unit 35 is normally operated based on the control of the driving control unit 27, and outputs the judgment result to the driving control unit 27. Whether or not the operation confirming control unit 35 is operated normally is performed so that, for example, a check signal for requesting a reply is transmitted from the operation confirming control appropriateness judging unit to the operation confirming control unit 35, and when there is a reply from the operation confirming unit 35, the operation confirming control appropriateness judging unit judges that the operation is normal, and when there is not a reply, the operation confirming control appropriateness judging unit judges that the operation is abnormal. Then, when the operation confirming control appropriateness judging unit judges that the operation is abnormal, the driving control unit 27 controls the indicator 25 to report that the operation is abnormal. At this time, the driving control unit 27 controls the communication unit 24 to transmit information indicating that the operation is abnormal to the console 6. Thereby, it is possible to confirm whether or not there is an abnormality of the operation confirming control unit 35 at a side of the console 6. In this way, preferably at least the driving control unit 27 and the operation confirming control unit 35 confirm one another's operation.

Further, in the present embodiment, described is the case that a charging device such as a cradle-for charging the battery charger 21 is used. However, the charging may be done by getting an electric power supply from an external power source by connecting a code for the electric power supply to a terminal of the radiation image detector. Further, the structure may be such that the charging is done in a state where the battery charger is taken out from the radiation image detector.

As above, the radiation image generating system of the present invention is useful for radiation image generation for which a cassette type radiation image detector is used, and especially suitable for performing radiation image generation by a radiation image detector which is moved to various locations.

The entire disclosure of a Japanese Patent Application No. Tokugan 2005-089186 filed on Mar. 25, 2005, including specifications, claims, drawings and summaries are incorporated herein by reference in their entirety.

What is claimed is:

1. A cassette type radiation image detector comprising:
a driving control unit to control a plurality of driving units; and
an operation confirming control unit to confirm an operation of the driving control unit,
wherein the cassette type radiation image detector obtains an image information.

2. The detector of claim 1, further comprising a battery as an electric power supplying source to the plurality of driving units, the driving control unit and the operation confirming control unit,
wherein the driving control unit comprises a battery residual quantity detecting unit to detect a residual quantity of the battery.

3. The detector of claim 1, wherein the plurality of driving units comprises a storing unit to store the image information, and
the driving control unit comprises a storing operation detecting unit to detect whether or not a storing operation of the storing unit is appropriate.

4. The detector of claim 1, wherein the plurality of driving units comprise a communication unit to communicate with an external device, and
the driving control unit comprises a communication operation detecting unit detecting whether or not a communication operation of the communication unit is appropriate.

5. The detector of claim 1, wherein the operation confirming control unit comprises a driving control appropriateness judging unit to judge whether or not the driving control unit is normally operated.

6. The detector of claim 5, wherein the driving control appropriateness judging unit judges whether the driving control unit is operated normally based on detected results of the battery residual quantity detecting unit, the storing operation detecting unit and the communication operation detecting unit of the driving control unit.

7. The detector of claim 5, further comprising a reporting unit to report based on a control of the operation confirming control unit,
wherein the operation confirming control unit controls the reporting unit to report an abnormality when the driving control appropriateness judging unit judges that the driving control unit is not operated normally.

8. The detector of claim 1, wherein the driving control unit comprises an operation confirming control appropriateness judging unit to judge whether or not the operation confirming control unit is operated normally.

9. The detector of claim 8, further comprising a reporting unit to report based on a control of the driving control unit,
wherein the driving control unit controls the reporting unit to report an abnormality when the operation confirming control appropriateness judging unit judges that the operation confirming control unit is not operated normally.

10. A radiation image detecting system comprising:
a cassette type radiation image detector to obtain an image information, having a driving control unit to control a plurality of driving units, and an operation confirming control unit to confirm an operation of the driving control unit; and
a console to control the cassette type radiation image detector.

11. The system of claim 10, wherein the operation confirming control unit controls the driving control appropriateness judging unit to transmit an information indicating an abnormality to the console, when the driving control appropriateness judging unit judges that the driving control unit is not operated normally.

12. The system of claim 10, wherein the driving control unit controls the operation confirming control appropriateness judging unit to transmit an information indicating an abnormality to the console, when the operation confirming control appropriateness judging unit judges that the operation confirming control unit is not operated normally.
